(19) United States
(12) Patent Application Publication
(10) Pub. No.: US 2026/0334829 A1
(43) Pub. Date: Oct. 29, 2026

(54) SUBCUTANEOUS IMPLANTABLE MULTI-ANALYTE ELECTROCHEMICAL SENSOR FOR CONTINUOUS pH AND CARBON DIOXIDE MONTIORING

(71) Applicant: Continuum Diagnostics, Irvine, CA (US)

(72) Inventors: Nicholas Xavier WILLIAMS, Washington, DC (US); Xun CHEN, Shanghai (CN); Pan ZHOU, Shanghai (CN); Lili ZHAO, Shanghai (CN)

(21) Appl. No.: 19/652,761

(22) Filed: Apr. 20, 2026

Related U.S. Application Data
(60) Provisional application No. 63/794,769, filed on Apr. 25, 2025.

(51) Int. Cl. A61B 5/1473; A61B 5/00; A61B 5/145

(52) U.S. Cl. CPC A61B 5/1473; A61B 5/1451; A61B 5/14517; A61B 5/14539; A61B 5/14546; A61B 5/4818; A61B 5/686; A61B 5/7275; A61B 5/742; A61B 5/746; A61B 2505/03; A61B 2505/05; A61B 2560/063; A61B 2562/12

(57) ABSTRACT

A subcutaneous implantable multi-analyte electrochemical sensor includes a flexible substrate, a first working electrode selective for hydrogen ions, a second working electrode selective for carbonate ions, and at least one reference electrode. Each working electrode includes a conductive layer, a conductive medium layer, an ion-selective membrane layer, and an outer membrane layer. The first and second working electrodes form respective measurement loops with the at least one reference electrode to generate electrical signals corresponding to hydrogen ion concentration and carbonate ion concentration in a biological fluid. A processor may calculate pH from the hydrogen-ion-related signal and derive a carbon dioxide concentration or partial pressure from the hydrogen-ion-related signal and the carbonate-ion-related signal. Methods of fabrication and monitoring systems using the sensor are also disclosed.

### CROSS-REFERENCE TO RELATED APPLICATIONS

This application claims the benefit of U.S. Provisional Patent Application No. 63/794,769 filed Apr. 25, 2025 which is incorporated herein in its entirety.

### FIELD OF THE INVENTON

The invention relates to implantable electrochemical sensors and, more particularly, to subcutaneous multi-channel sensors configured to continuously measure hydrogen ion concentration and carbon dioxide-related analytes in a biological fluid for dynamic monitoring of respiratory status and electrolyte-related parameters.

### BACKGROUND OF THE INVENTION

### Description of the Related Art

In rapidly changing clinical settings for critically ill patients, respiratory-function data such as pH and carbon dioxide must be collected frequently to ensure appropriate ventilation and therapeutic intervention. Conventional blood-gas monitoring typically relies on venous or arterial puncture to obtain blood samples that are analyzed using a benchtop blood-gas analyzer. In the early stages of care, when patient status can change quickly, blood-gas samples may be taken every hour or every other hour.

For intensive care unit patients and pediatric patients, repeated arterial or venous puncture imposes physiological burden and creates cumulative risk of infection and other adverse events. Even when blood-gas sampling is performed frequently, the resulting measurements still provide only snapshots of blood-gas values at isolated moments in time and may fail to capture rapid changes in respiratory function occurring between sampling events.

Continuous glucose monitoring systems demonstrated that minimally invasive, continuously operating implantable sensors can transform management of a physiological parameter that previously depended on repeated blood sampling. A comparable approach for blood-gas-related parameters could enable dynamic tracking of respiratory and electrolyte status while reducing patient discomfort and procedural risk.

A subcutaneously implantable sensor capable of continuously measuring pH and carbon dioxide-related analytes in interstitial fluid, blood, sweat, or other biological fluids would therefore address a significant clinical need. Such a sensor could support monitoring during intensive care, surgery, extracorporeal membrane oxygenation, mechanical ventilation, chronic respiratory-disease management, sleep-apnea management, and other settings in which respiratory status changes over time. The present invention addresses that need.

### SUMMARY OF THE INVENTION

The invention provides a subcutaneous implantable multi-analyte electrochemical sensor that includes a flexible insulating substrate, at least a first working electrode and a second working electrode, and at least one reference electrode. In certain embodiments, the sensor further includes at least one counter electrode. The electrodes may be arranged on a common surface of the substrate or on opposite surfaces of the substrate in different layout schemes.

A first working electrode includes a conductive layer, a conductive medium layer, an ion-selective membrane layer selective for hydrogen ions, and an outer membrane layer. A second working electrode includes a conductive layer, a conductive medium layer, an ion-selective membrane layer selective for carbonate ions, and an outer membrane layer. The first working electrode and the at least one reference electrode define a first electrical measurement loop, and the second working electrode and the at least one reference electrode define a second electrical measurement loop.

In use, the first working electrode generates a signal corresponding to hydrogen ion concentration, from which pH can be determined. The second working electrode generates a signal corresponding to carbonate ion concentration. By combining the measured hydrogen ion concentration and carbonate ion concentration using carbonate-equilibrium relationships, a carbon dioxide concentration or partial pressure can be derived.

In certain embodiments, the conductive medium layer includes carbon nanotubes dispersed in a solvent and applied in multiple layers by a dotting process. In certain embodiments, the hydrogen ion selective membrane includes hydrogen ion carrier I, potassium tetrakis(4-chlorophenyl)borate, o-nitrophenylpropionic acid, and polyurethane. In certain embodiments, the carbonate ion selective membrane includes carbonate ion carrier VII, cetyltrimethylammonium chloride, 2-ethylhexyl acrylate, and poly(vinyl chloride).

The invention further provides fabrication methods in which conductive layers are formed on the substrate by printing, conductive medium layers are applied by dotting, ion-selective membrane layers are applied in multiple coating steps with drying or curing intervals, and the completed working electrodes are coated with a biocompatible outer membrane by dip coating. The invention also provides monitoring systems and methods that use the implanted sensor together with a potentiostat circuit, processor, and optional wireless transmitter to provide near real-time monitoring, display, and alert generation.

### BRIEF DESCRIPTION OF THE DRAWINGS

FIG. 1 is a schematic diagram of a multi-channel sensor for pH and CO2.

FIG. 2 illustrates electrode layout schemes for a subcutaneous implantable pH and CO2 multi-channel sensor.

FIG. 3 is a schematic diagram of a first working electrode for a subcutaneous implantable pH and CO2 multi-channel sensor.

FIG. 4 is a schematic diagram of a second working electrode for a subcutaneous implantable pH and CO2 multi-channel sensor.

FIG. 5 illustrates a fabrication process of microelectrodes for a pH and CO2 multi-channel sensor.

FIG. 6 illustrates a fabrication process of membrane layers for a subcutaneous implantable pH and CO2 multi-channel sensor.

### DETAILED DESCRIPTION OF THE INVENTION

The invention provides a subcutaneous implantable multi-channel sensor for blood-gas analysis. The sensor includes a flexible substrate and at least two working electrodes, at least one reference electrode, and optionally at least one counter electrode disposed on the substrate. Each electrode is electrically independent on the substrate, with insulating structure separating adjacent conductive regions. A first working electrode and a second working electrode may be formed on the same plane of the substrate or on different planes of the substrate.

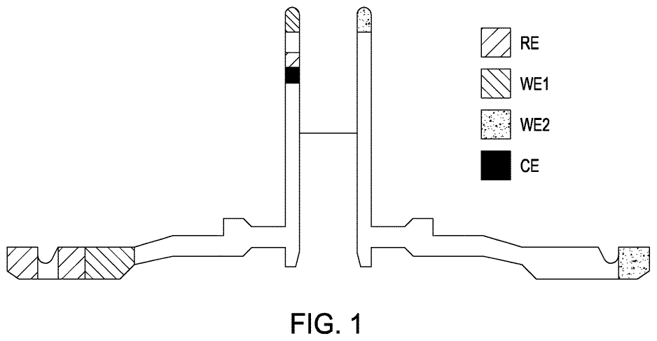

FIG. 1 schematically illustrates a representative multi-channel sensor architecture in which a flexible insulating substrate carries at least a first working electrode, a second working electrode, and at least one reference electrode, and optionally a counter electrode, arranged to support parallel electrochemical measurement loops. As shown, the sensing region is configured for subcutaneous placement so that the electrode structures contact a biological fluid such as interstitial fluid, and the individual electrode paths may be electrically coupled to external circuitry that measures potential differences corresponding to hydrogen ion concentration and carbonate-ion-related concentration.

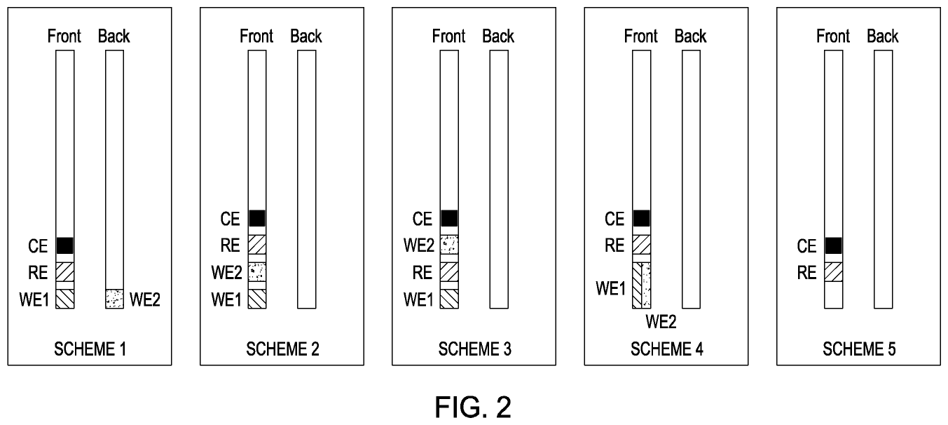

FIG. 2 illustrates several contemplated electrode layout schemes for the subcutaneous implantable multi-channel sensor. In some embodiments, the first working electrode, second working electrode, reference electrode, and optional counter electrode are arranged on a common surface of the substrate, while in other embodiments one or more working electrodes are disposed on an opposite surface to conserve space, improve isolation, or facilitate implantation geometry. The different schemes demonstrate that the invention is not limited to a single electrode pattern and that the front-side and back-side placement of the electrodes may be selected according to manufacturability, sensing stability, signal isolation, or desired analyte configuration.

A first working electrode is configured to generate an electrical potential difference responsive to hydrogen ion concentration after contact with tissue interstitial fluid, blood, sweat, or another biological fluid. The second working electrode is configured to generate an electrical potential difference responsive to carbonate ion concentration after contact with tissue interstitial fluid, blood, sweat, or another biological fluid. The first working electrode and the reference electrode define a first circuit group for measuring a first electrical potential signal, and a second working electrode and the reference electrode define a second circuit group for measuring a second electrical potential signal. The reference electrode may be shared by both circuit groups, or a separate reference electrode may be provided for each circuit group. In embodiments that include a counter electrode, the counter electrode may also be shared or separately assigned.

The first working electrode includes a conductive layer printed on the substrate through a printing process. A conductive medium layer, which in certain embodiments is a carbon nanotube solution, is applied on the conductive layer through a dotting process to improve conductivity. An ion-selective membrane layer selective for hydrogen ions is then applied. This membrane can block interfering small molecules while allowing hydrogen ions to participate in formation of an electrical potential difference relative to the reference electrode. Hydrogen ion concentration can be obtained through the Nernst equation, and pH can then be calculated based on hydrogen ion concentration.

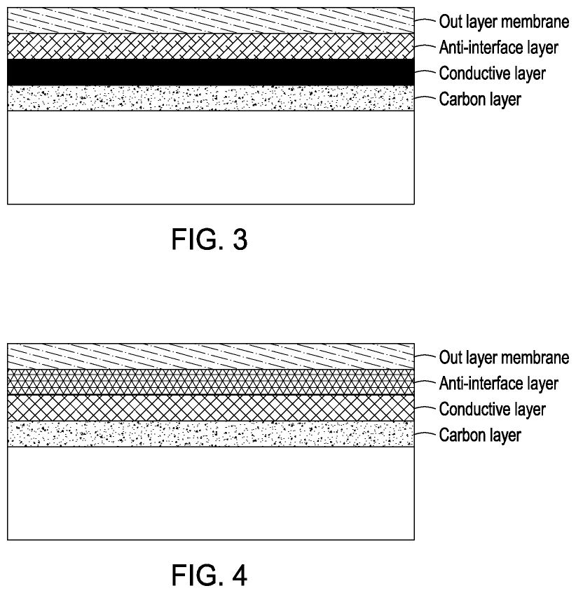

FIG. 3 schematically illustrates the layer structure of a first working electrode configured to sense hydrogen ions. In the illustrated embodiment, the first working electrode is formed in an outward sequence beginning with a conductive layer supported by the substrate, followed by a conductive medium layer that improves electron transfer and signal stability, followed by a hydrogen-ion-selective membrane, and then an outer membrane layer that provides biocompatibility and controls diffusion of analytes toward the underlying sensing layers. The figure thus shows how the first working electrode may be built as a layered electrochemical structure that selectively responds to hydrogen ion activity in the surrounding biological fluid.

In certain embodiments, the hydrogen ion selective membrane includes hydrogen ion carrier I, potassium tetrakis(4-chlorophenyl)borate, o-nitrophenylpropionic acid, and polyurethane. The outer membrane layer over the first working electrode may include polyurethane and functions to control analyte diffusion while providing biocompatibility.

The second working electrode includes a conductive layer printed on the substrate through a printing process and a conductive medium layer applied by dotting to improve conductivity. An ion-selective membrane layer selective for carbonate ions is applied over the conductive medium layer. This membrane can block interfering species while allowing measurement of carbonate-ion-related potential relative to the reference electrode. The resulting signal can be used to determine carbonate ion concentration. Combined with hydrogen ion concentration, a carbonate dissociation equilibrium relationship and a carbon dioxide partial-pressure relationship may be used to derive carbon dioxide concentration or partial pressure in the body.

FIG. 4 schematically illustrates the layer structure of a second working electrode configured to sense carbonate ions or a carbonate-ion-related parameter used to derive carbon dioxide concentration or partial pressure. In the illustrated embodiment, the second working electrode similarly includes a conductive layer on the substrate, a conductive medium layer, a carbonate-ion-selective membrane, and an outer membrane layer. The figure demonstrates that although the first and second working electrodes may share a common layered architecture, the ion-selective membrane chemistry can differ so that each electrode selectively generates a distinct electrical response for a different target analyte.

In certain embodiments, the carbonate ion selective membrane includes carbonate ion carrier VII, cetyltrimethylammonium chloride, 2-ethylhexyl acrylate, and poly(vinyl chloride). The outer membrane layer over the second working electrode may also include polyurethane and functions as a biocompatible diffusion-limiting membrane.

In one fabrication approach, the first layer of each working electrode is a conductive layer comprising carbon paste printed on the insulating substrate through a screen-printing process. A conductive medium layer solution is then applied by dotting. An ion-selective membrane reagent is subsequently applied to form an ion-selective membrane layer. After membrane formation, an outer membrane layer reagent is applied, including by immersion or dip coating, and the resulting structure is cured to form the completed working electrode.

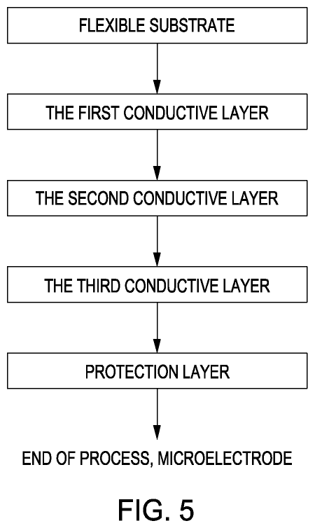

FIG. 5 illustrates an example fabrication process for forming the microelectrode structures of the sensor on the flexible substrate. In one representative process, conductive traces and electrode regions are first printed on the substrate, such as by screen printing, to define the working, reference, and optional counter electrodes. Conductive medium material may then be applied to selected working-electrode regions, after which the patterned substrate is allowed to dry or cure before further membrane-processing steps are performed. The figure therefore depicts a representative sequence for producing the base microelectrode architecture that later receives the analyte-selective membrane coatings.

In one example, the conductive medium layer of the first working electrode is composed of a lipophilic carbon nanotube solution. Lipophilic carbon nanotubes are dispersed in tetrahydrofuran solvent at a concentration of approximately 1 mg/mL. Approximately 2 uL of the dispersion is applied to the conductive layer through dotting. After drying at room temperature for approximately 10 minutes, another layer is applied. This process may be repeated for a total of approximately ten layers, and the structure may then be allowed to stand for approximately twelve hours so that the solvent evaporates completely.

In one example, the hydrogen ion selective membrane layer is prepared by dissolving approximately 1.1 mg of hydrogen ion selective carrier I, approximately 0.3 mg of potassium tetrakis(4-chlorophenyl)borate, approximately 65.8 mg of o-nitrophenylpropionic acid, and approximately 32.9 mg of polyurethane in approximately 1 mL of tetrahydrofuran to obtain an ion-selective membrane solution. The solution may be applied to the working electrode area through dotting three times with an interval of approximately twenty minutes between applications. After the third application, the sample may be transferred to a temperature-and humidity-controlled chamber at approximately 37 degrees C and approximately 50 percent humidity for approximately twenty-four hours to allow solvent evaporation and membrane formation.

In one example, the carbonate ion selective membrane layer is prepared by dissolving approximately 4.1 mg of carbonate ion carrier VII, approximately 1 mg of cetyltrimethylammonium chloride, approximately 50 uL of 2-ethylhexyl acrylate, and approximately 30 mg of poly(vinyl chloride) in approximately 1 mL of tetrahydrofuran. The solution may be applied to the working electrode area through dotting three times with an interval of approximately twenty minutes between applications, followed by curing at approximately 37 degrees C and approximately 50 percent humidity for approximately twenty-four hours.

The sensor may further include a counter electrode. In certain embodiments, the counter electrode comprises a conductive layer mainly composed of carbon paste printed directly on the insulating layer through a screen-printing process. The sensor may further include a reference electrode mainly composed of silver/silver chloride paste formed by screen printing. The reference electrode forms an electrical potential difference with first and second working electrodes, enabling determination of hydrogen ion and carbonate ion concentrations and, ultimately, pH and carbon dioxide-related values.

Additional working electrodes may be added to measure further analytes such as potassium ions, chloride ions, sodium ions, or other analytes using ion-selective layers tailored to those analytes. Those additional sensors may be fabricated using the same general procedure with a different ion-selective membrane composition for the relevant ion.

Afterwards, the outer membrane layer may be applied by dip coating. In one embodiment, the polymer for the outer membrane layer is a polyurethane including polydimethylsiloxane as a hydrophobic segment and polyethylene glycol as a hydrophilic segment at a molar ratio of approximately 3:2. The polyurethane may be dissolved in ethanol to obtain a solution having a solids content of approximately 150 mg/mL. The implantable portion of the sensor may be dipped into the coating solution approximately six times, each dip lasting approximately four seconds, and then cured at approximately 37 degrees C and approximately 50 percent humidity for approximately twenty-four hours.

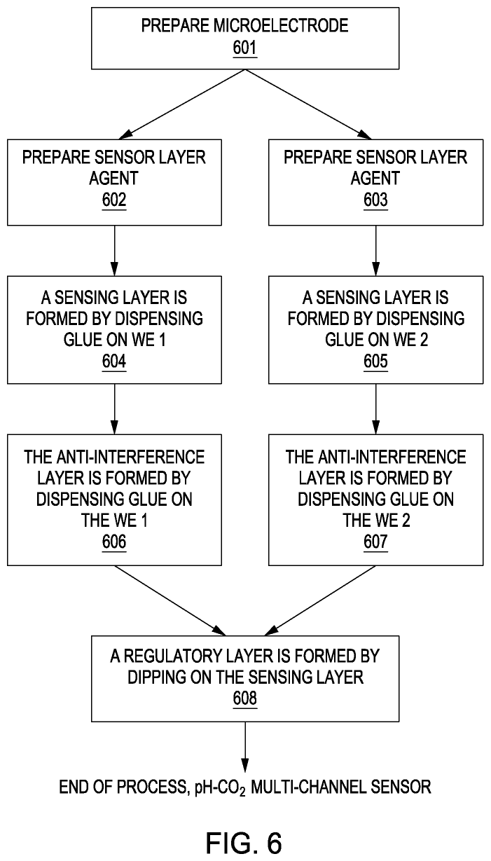

FIG. 6 illustrates a membrane-layer fabrication workflow having eight process blocks followed by a final output. In a first block 601, a previously fabricated microelectrode structure is prepared as the base sensor platform on which membrane materials are subsequently deposited. In a second block 602, a first sensor-layer agent is prepared for the first working electrode, and in preferred embodiments this agent comprises a hydrogen-ion-selective membrane formulation containing one or more hydrogen ion carriers, a lipophilic salt, a plasticizer or solvent system, and a polymer matrix. In a third block 603, a second sensor-layer agent is prepared for the second working electrode, and in preferred embodiments this agent comprises a carbonate-ion-selective membrane formulation containing a carbonate ion carrier, an ion exchanger, a plasticizer, and a polymer matrix. In a fourth block 604, a sensing layer is formed on working electrode 1 (WE1) by dispensing or dotting the first sensor-layer agent onto the designated electrode region in one or more coating applications. In a fifth block 605, a corresponding sensing layer is formed on working electrode 2 (WE2) by dispensing or dotting the second sensor-layer agent onto the designated electrode region in one or more coating applications. In a sixth block 606, an anti-interference layer is formed on WE1 by dispensing a further coating over the previously formed sensing layer so that interfering species are reduced while the target hydrogen ions remain measurable. In a seventh block 607, an anti-interference layer is formed on WE2 by dispensing a further coating over the previously formed sensing layer so that interfering species are reduced while carbonate-related species remain measurable. In an eighth block 608, a regulatory layer is formed by dipping the sensing region into an outer-layer coating solution, thereby forming a diffusion-regulating and biocompatible membrane over the previously coated sensing layers. The final output of the FIG. 6 process is an end-of-process pH-CO2 multi-channel sensor in which the microelectrode base, the analyte-selective sensing layers, the anti-interference layers, and the outer regulatory layer have been assembled into a completed implantable sensing structure suitable for calibration, packaging, and subcutaneous use.

At least five or more electrode-distribution schemes are contemplated. In one preferred scheme, the implantable part includes a counter electrode, a reference electrode, and one working electrode on the front side, with the other working electrode printed on the back side. In other schemes, the counter electrode, reference electrode, and both working electrodes may all be on the front side in different relative arrangements. In another scheme, the front side may include the counter electrode and reference electrode while a working electrode extends on the back side.

The sensor may be dimensioned for insertion through a needle-based applicator into subcutaneous tissue so that the sensing region resides in interstitial fluid. A potentiostat circuit may be electrically coupled to the sensor to measure electrical potential differences relative to the at least one reference electrode. A processor may calculate pH from the first signal and carbonate concentration from the second signal, and may derive a carbon dioxide concentration or partial pressure using the measured values and an equilibrium relationship. In some embodiments, the measured or derived values are transmitted wirelessly to an external receiver for display in near real time. Alerts may be generated when values exceed thresholds or change at rates indicative of clinical deterioration.

Although illustrative embodiments are described, the invention is not limited to the specific materials, geometries, arrangements, analyte combinations, or process conditions expressly set forth herein. Variations, substitutions, and modifications consistent with the disclosed principles may be employed.

What is claimed is:

1. An implantable multi-analyte electrochemical sensor, comprising:
an insulating substrate having a first surface and a second surface;
a first working electrode disposed on the substrate, the first working electrode comprising, in sequence from the substrate outward, a first conductive layer, a first conductive medium layer, a first ion-selective membrane layer selective for hydrogen ions, and a first outer membrane layer;
a second working electrode disposed on the substrate, the second working electrode comprising, in sequence from the substrate outward, a second conductive layer, a second conductive medium layer, a second ion-selective membrane layer selective for carbonate ions, and a second outer membrane layer; and
at least one reference electrode disposed on the substrate,
wherein the first working electrode and the at least one reference electrode form a first circuit loop configured to generate a first electrical signal responsive to concentration of hydrogen ions in a biological fluid, and the second working electrode and the at least one reference electrode form a second circuit loop configured to generate a second electrical signal responsive to concentration of carbonate ions in the biological fluid.

2. The sensor of claim 1, wherein the biological fluid is selected from the group consisting of interstitial fluid, venous blood, arterial blood, and sweat.

3. The sensor of claim 1, wherein the first conductive medium layer and the second conductive medium layer each comprise carbon nanotubes dispersed in a solvent and applied in a plurality of layers.

4. The sensor of claim 3, wherein the solvent comprises tetrahydrofuran.

5. The sensor of claim 1, wherein the first ion-selective membrane layer comprises hydrogen ion carrier I, potassium tetrakis(4-chlorophenyl)borate, o-nitrophenylpropionic acid, and polyurethane.

6. The sensor of claim 1, wherein the second ion-selective membrane layer comprises carbonate ion carrier VII, cetyltrimethylammonium chloride, 2-ethylhexyl acrylate, and poly(vinyl chloride).

7. The sensor of claim 1, wherein the first outer membrane layer and the second outer membrane layer each comprise polyurethane configured to control analyte diffusion and provide biocompatibility.

8. The sensor of claim 1, further comprising at least one counter electrode disposed on the substrate.

9. The sensor of claim 1, wherein the at least one reference electrode comprises a silver/silver chloride conductive layer.

10. The sensor of claim 1, wherein the first working electrode, the second working electrode, and the at least one reference electrode are disposed on the first surface of the substrate.

11. The sensor of claim 1, wherein the first working electrode is disposed on the first surface of the substrate and the second working electrode is disposed on the second surface of the substrate.

12. The sensor of claim 1, further comprising at least one additional working electrode disposed on the substrate and having an ion-selective membrane layer selective for an additional analyte selected from the group consisting of potassium ions, chloride ions, and sodium ions.

13. A method of fabricating an implantable multi-analyte electrochemical sensor, comprising:
providing a flexible insulating substrate;
forming a first conductive layer and a second conductive layer on the substrate by a printing process;
forming at least one reference electrode on the substrate;
applying a first conductive medium layer onto the first conductive layer and a second conductive medium layer onto the second conductive layer;
applying a first ion-selective membrane solution onto the first conductive medium layer to form a first ion-selective membrane layer selective for hydrogen ions;
applying a second ion-selective membrane solution onto the second conductive medium layer to form a second ion-selective membrane layer selective for carbonate ions; and coating the first ion-selective membrane layer and the second ion-selective membrane layer with an outer membrane layer.

14. The method of claim 13, wherein the printing process comprises screen printing.

15. The method of claim 13, wherein applying the first conductive medium layer and the second conductive medium layer comprises applying a carbon nanotube dispersion in a plurality of discrete dotting layers with drying intervals between successive layers.

16. The method of claim 13, wherein applying the first ion-selective membrane solution and the second ion-selective membrane solution comprises applying each solution in a plurality of coating steps separated by drying intervals and thereafter curing the coated substrate in a temperature-and humidity-controlled chamber.

17. The method of claim 13, wherein coating the first ion-selective membrane layer and the second ion-selective membrane layer with the outer membrane layer comprises dip coating an implantable portion of the sensor in a polyurethane solution a plurality of times.

18. A method of continuously monitoring at least two analyte-related values in a biological fluid of a subject, comprising:
subcutaneously implanting an electrochemical sensor into the subject, the sensor comprising a first working electrode having a first ion-selective membrane selective for hydrogen ions, a second working electrode having a second ion-selective membrane selective for carbonate ions, and at least one reference electrode;
measuring a first electrical potential between the first working electrode and the at least one reference electrode to obtain a first signal correlating with hydrogen ion concentration in the biological fluid;
measuring a second electrical potential between the second working electrode and the at least one reference electrode to obtain a second signal correlating with carbonate ion concentration in the biological fluid;
calculating a pH value from the first signal; and
deriving a carbon dioxide concentration or partial pressure from the pH value and the second signal using a carbonate-equilibrium relationship.

19. The method of claim 18, further comprising wirelessly transmitting data corresponding to at least one of the first signal, the second signal, the pH value, or the carbon dioxide concentration or partial pressure to an external receiver for display in near real time.

20. The method of claim 18, further comprising generating an alert when the pH value or the carbon dioxide concentration or partial pressure exceeds a predetermined threshold or exhibits a rate of change exceeding a predetermined rate.

21. The method of claim 18, wherein the subject is undergoing at least one of mechanical ventilation, extracorporeal membrane oxygenation, surgery, treatment in an intensive care unit, monitoring of chronic respiratory disease, or monitoring for sleep apnea.

22. A continuous monitoring system, comprising:
a subcutaneously implantable electrochemical sensor comprising a flexible insulating substrate, a first working electrode having a first ion-selective membrane layer selective for hydrogen ions, a second working electrode having a second ion-selective membrane layer selective for carbonate ions, and at least one reference electrode;
a potentiostat circuit operably connected to the first working electrode, the second working electrode, and the at least one reference electrode, the potentiostat circuit configured to measure a first electrical potential corresponding to hydrogen ion concentration and a second electrical potential corresponding to carbonate ion concentration; and
a processor operably connected to the potentiostat circuit, the processor configured to calculate a pH value from the first electrical potential and to derive a carbon dioxide concentration or partial pressure from the first electrical potential and the second electrical potential.

23. The system of claim 22, further comprising a wireless transmitter operably connected to the processor and an external receiver configured to receive data from the wireless transmitter and display values or trends corresponding to at least one of pH, carbonate concentration, or carbon dioxide concentration or partial pressure.

24. The system of claim 22, wherein the sensor is dimensioned for insertion via a needle-based applicator and the potentiostat circuit is housed in a wearable patch configured to overlie the implanted sensor.
